(19) United States
(12) Patent Application Publication
(10) Pub. No.: US 2024/0115767 A1
(43) Pub. Date: Apr. 11, 2024

(54) ALTERNATING PRESSURE AND VACUUM SYSTEM FOR DECELLULARIZING A BONE MATRIX

(71) Applicant: EPIBONE, INC., Brooklyn, NY (US)

(72) Inventors: Sarindr BHUMIRATANA, Oceanside, NY (US); Eric Meade JEFFRIES, Warrington, PA (US); Keith YEAGER, Springfield, NJ (US)

(73) Assignee: EPIBONE, INC., Brooklyn, NY (US)

(21) Appl. No.: 17/963,304

(22) Filed: Oct. 11, 2022

(51) Int. Cl. A61L 27/36; C12N 5/00

(52) U.S. Cl. CPC A61L 27/3691; A61L 27/3608; A61L 27/3687; C12N 5/0081

(57) ABSTRACT

An alternating vacuum and pressure system for decellularizing a bone matrix includes piping having a first end open to atmospheric pressure and a second end open to atmospheric pressure, and a subset of piping that forms a loop, a chamber in fluid, at least one heating element configured to heat the piping, a pump, and a plurality of valves. The plurality of valves can be selectively opened or closed to form one of a plurality of configurations, including a pressure configuration in which operation of the pump in a first direction causes a fluid within the piping to travel toward the chamber such that pressure is applied to the chamber and a vacuum configuration in which operation of the pump in a second direction causes the fluid within the pipe to travel away from the chamber such that a vacuum is created on the chamber.

### TECHNICAL FIELD

The present invention relates generally to decellularizing a bone matrix, and, more particularly, to systems and methods of decellularizing a bone matrix using alternating pressure and vacuum.

### BACKGROUND

Alternating cycles of high pressure and vacuum, when used in conjunction with appropriate solutions, can be used to decellularize bone matrices or other tissue. These decellularized bone matrices can be used as scaffolds, seeded with human mesenchymal stem cells (hMSCs), and cultivated to create bone grafts.

### SUMMARY

Embodiments of the present invention address and overcome one or more of the shortcomings and drawbacks of the prior art, by providing an alternating pressure and vacuum system and a method of decellularizing a bone matrix.

According to some embodiments, an alternating vacuum and pressure system for decellularizing a bone matrix can include piping having a first end open to atmospheric pressure and a second end open to atmospheric pressure, and a subset of piping that forms a loop, a chamber in fluid, at least one heating element configured to heat the piping, a pump, and a plurality of valves. The plurality of valves can be selectively opened or closed to form one of a plurality of configurations, including a pressure configuration in which operation of the pump in a first direction causes a fluid within the piping to travel toward the chamber such that pressure is applied to the chamber and a vacuum configuration in which operation of the pump in a second direction causes the fluid within the pipe to travel away from the chamber such that a vacuum is created on the chamber.

Various enhancements, refinements, and other modifications may be made to the system discussed above. For example, in some embodiments, the plurality of valves can include a loop valve, wherein closure of the loop valve prevents the fluid within the piping from circulating through the loop, and wherein the loop valve is closed in the pressure configuration and the vacuum configuration. In some embodiments, the plurality of valves can further include a pump valve, wherein closure of the pump valve prevents pumping of a fluid within the piping, and wherein the pump valve is closed after pressure is established in the pressure configuration and after vacuum is established in the vacuum configuration. In some embodiments, the plurality of valves can further include an air valve, wherein closure of the air valve closes the first end to atmospheric pressure, and wherein the air valve is closed in the pressure configuration and the vacuum configuration. In some embodiments, the plurality of valves can further include a fill valve, wherein closure of the fill valve prevents a filling fluid from being drawn into the piping, and wherein the fill valve is closed in the pressure configuration and the vacuum configuration. Additionally, in some embodiments, the plurality of valves can further include a drain valve, wherein closure of the drain valve closes the second end to atmospheric pressure prevents the fluid within the piping from exiting the piping through the second end, and wherein the drain valve is closed in the pressure configuration and the vacuum configuration.

Further, in some embodiments, the plurality of configurations can further include a recirculation configuration, wherein, in the recirculation configuration, operation of the pump causes fluid to circulate through the loop, and wherein, in the recirculation configuration, the loop valve and the pump valve are open, and the air valve, the fill valve, and the drain valve are closed. In some embodiments, the plurality of configurations can further include a balance configuration, wherein in the balance configuration a pressure of the fluid within the piping will balance, wherein, in the balance configuration, the air valve, the loop valve, and the pump valve are open, and the fill valve and the drain valve are closed. In some embodiments, the plurality of configurations can further include a drain configuration, wherein, in the drain configuration, the fluid within the piping exits the piping through the second end, and wherein, in the drain configuration, the air valve, the loop valve, the pump valve, and the drain valve are open. Additionally, in some embodiments, the plurality of configurations can further include a fill configuration, wherein, in the fill configuration, operation of a fill pump causes a filling fluid to travel from a fluid filling source into the piping, wherein, in the fill configuration, the fill valve, the pump valve, the loop valve, and the air valve are open, and the drain valve is closed.

Further, in some embodiments, the fluid filling source can be one of a plurality of tanks including a water fluid tank comprising water, an alcohol fluid tank comprising isopropanol, an acid fluid tank comprising ethylenediaminetetraacetic acid (EDTA), and a detergent fluid tank comprising a detergent solution. In some embodiments, the alternating vacuum and pressure system can further include a water fluid tank comprising water, an isopropanol fluid tank comprising isopropanol, an EDTA fluid tank comprising EDTA, and a detergent fluid tank comprising a detergent solution.

Further, in some embodiments, the chamber can include a basket comprising stainless steel mesh, and wherein the basket is configured to contain the bone matrix. In some embodiments, the chamber can further include a lid for the basket, wherein the lid comprises stainless steel mesh.

Further, in some embodiments, the at least one heating element can include a chamber heater configured to heat the chamber. In some embodiments, the at least one heating element can include a heating column attached to an exterior surface area of a portion of the piping, wherein the heating column is configured to heat the piping, which will in turn heat the fluid within the piping.

According to another aspect of the present invention, a method of decellularizing a bone matrix, the method includes providing a bone matrix in a piping forming a loop, wherein the piping comprises a pump, performing, for each of a plurality of fluids, a plurality of iterations, and draining the respective fluid from the loop. Performing a plurality of iterations can include filling the loop with a respective fluid of the plurality of fluids, operating the pump in a first direction to cause the respective fluid to achieve a positive pressure on the bone matrix, and operating the pump in a second direction to cause the respective fluid to achieve a vacuum on the bone matrix.

Various enhancements, refinements, and other modifications may be made to the method discussed above. For example, in some embodiments, performing, for each of a plurality of fluids, a plurality of iterations can include a first plurality of iterations configured to remove bulk debris from the bone matrix, a second plurality of iterations to remove lipids from the bone matrix, and a third plurality of iterations to remove cellular artifacts from the bone matrix.

In some embodiments, the plurality of fluids can include a detergent solution, a water solution, an alcohol solution, and an EDTA solution. In some embodiments, performing, for each of the plurality of fluids, the plurality of iterations can include performing, for the detergent solution, the first plurality of iterations, filling the loop with a first water solution in response to performing the first plurality of iterations, circulating the first water solution through the loop in response to filling the loop with a first water solution, draining the first water solution from the loop in response to circulating the first water solution through the loop, performing, for an isopropanol solution, the second plurality of iterations in response to draining the first water solution, filling the loop with a second water solution in response to performing the second plurality of iterations, circulating the second water solution through the loop in response to filling the loop with a second water solution, draining the second water solution from the loop in response to circulating the second water solution through the loop, performing, for the EDTA solution, the third plurality of iterations in response to draining the second water solution, filling the loop with a third water solution in response to performing the third plurality of iterations, circulating the third water solution through the loop in response to filling the loop with a third water solution, and draining the third water solution from the loop in response to circulating the third water solution through the loop.

Additional features and advantages of the invention will be made apparent from the following detailed description of illustrative embodiments that proceeds with reference to the accompanying drawings.

### BRIEF DESCRIPTION OF THE DRAWINGS

The foregoing and other aspects of the present invention are best understood from the following detailed description when read in connection with the accompanying drawings. For the purpose of illustrating the invention, there are shown in the drawings embodiments that are presently preferred, it being understood, however, that the invention is not limited to the specific instrumentalities disclosed. Included in the drawings are the following Figures:

FIG. 1 is a piping and instrumentation diagram of an exemplary embodiment of an alternating pressure and vacuum system;

FIG. 2 is a flow chart of an exemplary embodiment of a method of decellularizing a bone matrix; and

FIG. 3 is a flow chart of an exemplary embodiment of decellularizing a bone matrix; and

FIG. 4 is a photograph of an exemplary bone matrix chamber with a basket.

### DETAILED DESCRIPTION

The system disclosed herein can create alternating cycles of high pressure and vacuum that can be used in conjunction with the appropriate solutions to decellularize one or more highly porous bone matrices. In some embodiments, the system can be used with a detergent solution to remove the bulk of the debris from the bone matrix. The detergent solution can be periodically swapped with fresh detergent solution to reduce saturation of the detergent solution. In some embodiments, the detergent solution can be validated to reduce any potential viral load contaminating the raw material when temperature is maintained at 35° C. or greater. The system can also be used with Isopropanol (99%) to remove remaining lipids from and reduce the viral load of the bone matrix. In addition, the system can be used with an ethylenediaminetetraacetic acid (EDTA) solution to further remove residual DNA and other cellular artifacts. The system can also be used with a water solution to perform one or more water rinses and water washes to dilute and remove residual cleaning solution following the system's use with each of the above solutions. In some embodiments, a pair of coupled pistons driven by compressed air can be used to generate pressure and vacuum. In other embodiments, a pump can be used instead, for example, a pharmaceutical grade rotary lobe pump made of stainless steel and resistant gaskets, which can reduce risk of carry-over contamination from the device.

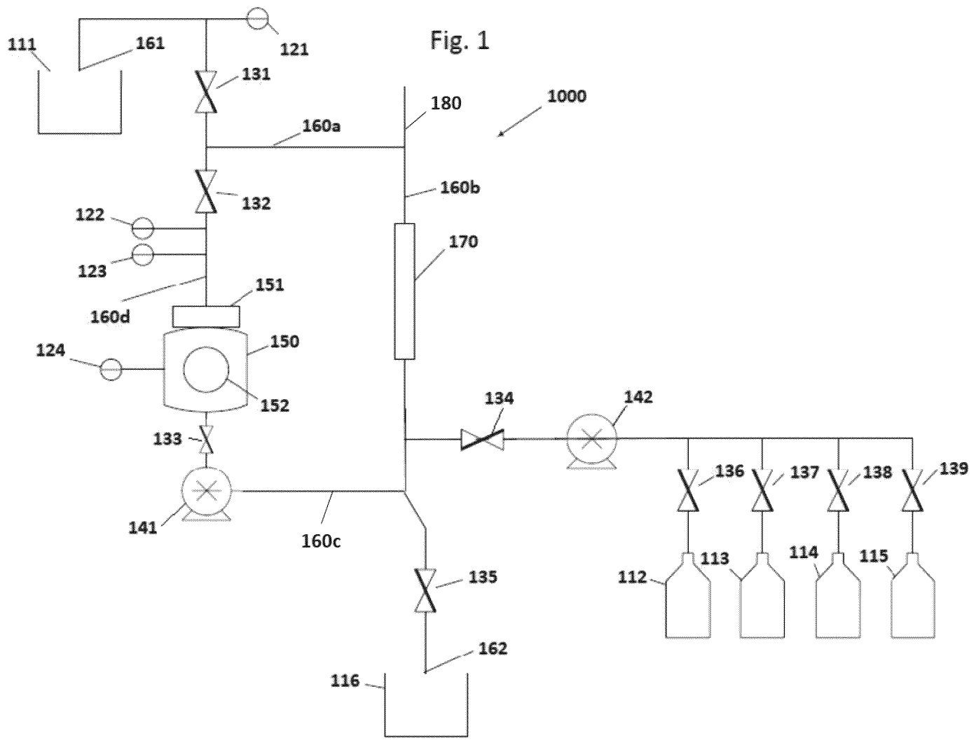

FIG. 1 is a piping and instrumentation diagram of an exemplary embodiment of an alternating vacuum and pressure (“AVAP”) system 1000. In this embodiment, the AVAP system 1000 comprises piping, a bone matrix chamber 150, a heating element, a pressure and vacuum pump 141, a fill pump 142, and a plurality of valves. The configuration of these and other elements enables the AVAP system 1000 to alternatively apply a high pressure and vacuum on the bone matrix chamber 150 to decellularize a bone matrix in the bone matrix chamber 150.

As shown in FIG. 1, the piping can have a first end 161 and a second end 162 that are each open to the atmosphere. A subset of the piping can also form a loop 160a-d. In some embodiments, the loop is formed by two vertically extending pipes 160b and 160d attached to and in fluid communication with two horizontal pipes 160a and 160c. The bone matrix chamber 150 can be attached to and in fluid communication with one of the vertically extending pipes 160b and 160d. In some embodiments, one of the two vertically extending pipes 160b and 160d can extend upward past the top horizontal pipe 160a and terminate at the first end 161, as illustrated in FIG. 1. In some embodiments, the piping extends beneath the bottom horizontal pipe 160c and terminates at the second end 162. As used herein, the term “piping” can include piping of any suitable material including flexible tubing.

As shown in FIG. 1, the AVAP system 1000 can include a bone matrix chamber 150. The bone matrix chamber 150 is configured to contain a bone matrix to be decellularized by the AVAP system 100. In some embodiments, the bone matrix chamber 150 is a pipe tee that forms a part of the piping and has its third end capped with a sight glass 152 so that the bone matrix within can be viewed. The basket 153 can be configured to hold the bone matrix. Using a basket 153 within the bone matrix chamber 150 can facilitate insertion and removal of the bone matrix. The basket 153 can be made of a porous material, such as a stainless steel mesh, so that the bone matrix within the basket 153 can be in contact with the fluid inside the piping 160. The basket 153 can also include a lid 154 made of a porous material, such as a stainless steel mesh.

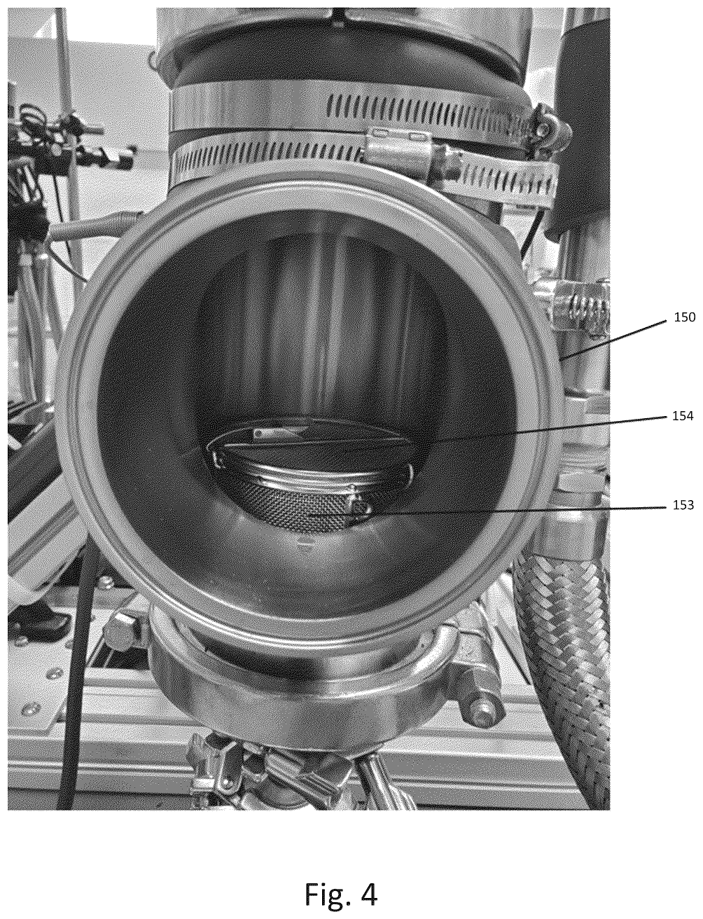

In some embodiments, the bone matrix chamber 150 can include a basket 153 removably attached within the bone matrix chamber 150, as shown in FIG. 4. In an embodiment, the basket 153 can be held in place within the bone matrix chamber 150 because its top face and bottom face are larger than the diameter of the piping above and below the bone matrix chamber 150, respectively. In other words, the basket 153 remains in the bone matrix chamber 150 because it is too big to travel out of the bone matrix chamber 150. However, as one of ordinary skill in the art will appreciate, the subject matter disclosed herein is not so limited. Instead, any suitable means for securing the basket 153 within the bone matrix chamber 150 are possible.

The AVAP system 1000 can also include at least one heating element. In some embodiments, the AVAP system 1000 can include a plurality of heating elements. The embodiment of FIG. 1 includes two heating elements: a bone matrix chamber heating element 151 and a column heating element 170. The bone matrix chamber heating element 151 is connected to the bone matrix chamber 150 and configured to heat the bone matrix chamber 150. The column heating element 170 is configured to attach to an external surface of the piping 160 and heat the piping, which will in turn heat the fluid within the piping 160. In some embodiments, the column heating element 170 wraps a section of the piping 160. In some embodiments, the heating elements can be configured to heat the fluid within the piping to a temperature above 37° C. to solubilize lipids of the bone matrix. For example, in some embodiments, the heating element can control the temperature of the fluid within the piping 160 between 35° C. and 45° C. In some embodiments, the AVAP system 1000 can further include temperature sensors and temperature and conductivity sensors, such as an inline temperature and conductivity sensor 122, an inline pressure sensor 123, and a heat surface temperature sensor 124, as shown in FIG. 1. A conductivity sensor can be used to sense conductivity. The conductivity can provide insight into the saturation of the fluid and can be used to ensure removal of the fluid, including, for example, an EDTA solution.

The AVAP system 1000 can include a pressure and vacuum pump 141. The pressure and vacuum pump 141 can be configured to pump the fluid within the piping 160 in one direction apply pressure to the bone matrix chamber and to pump the fluid within the piping 160 in the opposite direction to establish a vacuum on the bone matrix chamber 150. For example, the pressure and vacuum pump 141 can be configured to pump the fluid in a clockwise direction to apply pressure to the bone matrix chamber 150, and pump the fluid in a counter-clockwise direction to establish a vacuum on the bone matrix chamber 150. It will be understood that use of clockwise and counter-clockwise here is for illustrating the directional flow in the example piping and instrumentation diagram of FIG. 1, and is not intended to be limiting in any way to which direction the fluid can be pumped in an actual setting. In some embodiments, the pressure and vacuum pump 141 can pump a fluid towards the bone matrix chamber 150 such that a pressure of 75 psi gauge, 90 psi absolute is applied to the bone matrix chamber 150 and can pump a fluid away from the bone matrix chamber 150 such that more than an 80% vacuum is applied to the bone matrix chamber 150. In some embodiments, a rotary lobe pump can be used for the pressure and vacuum pump 141; however, one of ordinary skill in the art will appreciate that other pumps can be used with the system.

The AVAP system 1000 can also include a fill pump 142. The fill pump 142 can be configured to pump fluid from a reagent tank into the piping 160 when the fill valve 134 is open. In some embodiments, the fill pump 142 can be a peristaltic pump, but as one of ordinary skill in the art will appreciate, other types of pumps can be used.

The AVAP system 1000 can also include a plurality of valves. In some embodiments, the AVAP system 1000 can include an air valve 131, a loop valve 132, a pump valve 133, a fill valve 134, and a drain valve 135. These valves can be selectively opened or closed to place the AVAP system 1000 in one of a plurality of operating configurations. In an embodiment, the plurality of configurations can include a fill configuration, a balance configuration, a drain configuration, a recirculation configuration, a vacuum configuration, and a pressure configuration.

In some embodiments, the AVAP system 1000 can include an air pipe 180. The air pipe 180 can be a closed region of pipe that can trap an air pocket. In some embodiments, the air pipe 180 can be located on one side of the loop 160a-160d and the bone matrix chamber 150 can located on an opposite side of the loop 160a-160d, separated from the air pipe 180 by the loop valve 132 and the pump valve 133, as illustrated in FIG. 1. The air pipe 180 can contain air that can be expanded or compressed during the pressure and vacuum configurations. Because air is more expandable and more compressible than liquid, the presence of air in the air pipe 180 can facilitate greater generation of pressure and vacuum in the bone matrix chamber 150.

In some embodiments, the AVAP system 1000 can be arranged in a vertical set up in which the two vertically extending pipes 160b and 160d vertically extend. In this setup, the piping and connectors can be generally arranged vertically. The bone matrix chamber 150 can be on one side of the loop 160a-160d. The fill sensor 121 can be located at a higher elevation than the air valve 131 and the inlet of the fill valve 134. The air pipe 180 can be located at a higher elevation than the fill sensor 121. The benefit of vertical set up is that it can allow for controllable air removal through the air valve 131 and exit out through a first end 161 as fluid is being added to the AVAP system or during a degassing process.

To put the AVAP system 1000 in the fill configuration, the air valve 131, the loop valve 132, the pump valve 133, and the fill valve 134 are opened, and the drain valve 135 is closed. With the AVAP system 1000 in the fill configuration, operation of the fill pump 142 will draw fluid into the piping 160. In some embodiments, the AVAP system 1000 can further comprise a fill sensor 121 to sense the level of fluid within the piping 160. If AVAP system 1000 is overfilled, excess fluid can exit through first end 161. In some embodiments, the AVAP system 1000 can also include an overflow tank 111 to capture excess fluid exiting the first end 161.

The AVAP system 1000 can draw fluid from a reagent tank. In some embodiments, the AVAP system 1000 can include a water fluid tank 112, an alcohol fluid tank 113, an acid fluid tank 114, and a detergent fluid tank 115, each with a corresponding valve, 136, 137, 138, and 139, respectively. The alcohol fluid tank 113 can include, for example, isopropanol (e.g., 70%-100% isopropanol) for removing lipids from a bone matrix. However, the subject matter disclosed herein is not so limited. Instead, as one of ordinary skill in the art will appreciate, other alcohols may be used, including, for example, ethanol. The acid fluid tank 114 can include, for example, EDTA for removing residual DNA and other cellular artifacts from the bone matrix. Further, in some embodiments, the acid fluid tank 114 can include, for example EDTA and Tris Base. Use of the term “acid fluid tank” does not necessarily mean that the fluid stored in the tank requires or necessarily has a pH of less than 7. For example, in some embodiments, the prepared EDTA solution can be slightly basic. The acid fluid tank, however, does store the fluid that can be used to remove residual DNA from the decellularize bone matrices or other tissue, and EDTA is used here as an example. To fill the AVAP system with water, for example, the AVAP system is put into a fill configuration, the water tank valve 136 is opened, and the fill pump 142 is operated to pump water from the water fluid tank 112 into the piping 160.

To put the AVAP system 1000 in the balance configuration, the air valve 131, the loop valve 132, and the pump valve 133, are opened, and the fill valve 134 and the drain valve 135 are closed. With the AVAP system 1000 in the balance configuration, the pressure in the system will balance with atmospheric pressure.

To put the AVAP system 1000 in the recirculation configuration, the loop valve 132 and the pump valve 133 are opened, and the air valve 131, the fill valve 134, and the drain valve 135 are closed. With the AVAP system 1000 in the recirculation configuration, operation of pressure and vacuum pump 141 will cause the fluid within the piping 160 to circulate through the loop 160a-160d. This operation can be used to recirculate fluid, perform a wash cycle, or perform a rinse cycle.

To put the AVAP system 1000 in the drain configuration, the air valve 131, the loop valve 132, the pump valve 133, and the fill valve 134 are open, and the drain valve 135 is closed. With the AVAP system 1000 in the drain configuration, fluid will exit the piping 160. In some embodiments, the AVAP system 1000 can also include a drain tank 116 to capture fluid draining from the piping 160.

To put the AVAP system 1000 in the vacuum configuration, the pump valve 133 is opened and the air valve 131, the loop valve 132, the fill valve 134, and the drain valve 135 are closed. With the AVAP system 1000 in the vacuum configuration, operation of the pressure and vacuum pump 141 in one direction, for example, a forward or clockwise direction, will cause the fluid within the piping 160 to be drawn away from the bone matrix chamber 150, creating a vacuum on the bone matrix chamber 150. Because the loop valve 132 and the air valve 131 are each closed, operation of the pressure and vacuum pump 141 will not remove a significant amount of fluid from the bone matrix chamber 150. After a vacuum on the bone matrix chamber 150 is established, the pump valve 133 is closed and the pressure and vacuum pump 141 is shut off.

Like the vacuum configuration, to put the AVAP system 1000 in the pressure configuration, the pump valve 133 is opened and the air valve 131, the loop valve 132, the fill valve 134, and the drain valve 135 are closed. With the AVAP system 1000 in the pressure configuration, operation of the pressure and vacuum pump 141 in the other direction, for example, a backwards or counter-clockwise direction, will cause the fluid within the piping 160 to be pumped towards the bone matrix chamber 150, creating pressure on the bone matrix chamber 150. Because the loop valve 132 and the air valve 131 are each closed, operation of the pressure and vacuum pump 141 will not remove a significant amount of fluid from the bone matrix chamber 150. After pressure on the bone matrix chamber 150 reaches a predetermined level, the pump valve 133 is closed and the pressure and vacuum pump 141 is shut off.

While the foregoing example describes the pressure and vacuum pump 141 as operating in a forward or clockwise direction to establish a vacuum on the bone matrix chamber 150 and operating in a backwards or counter-clockwise direction to put the bone matrix chamber 150 under pressure, the subject matter disclosed herein is not so limited. Instead, in some embodiments, the pressure and vacuum pump 141 can operate in a backward or counter-clockwise direction to establish a vacuum on the bone matrix chamber 150 and operate in a forward or clockwise direction to put the bone matrix chamber 150 under pressure.

FIG. 1 illustrates where the air valve 131, the loop valve 132, the pump valve 133, the fill valve 134, and the drain valve 135 can be located. However, the subject matter disclosed herein is not so limited. Instead, as one of ordinary skill in the art will appreciate, the location of the valves on the piping can be changed as long as the above configurations are possible.

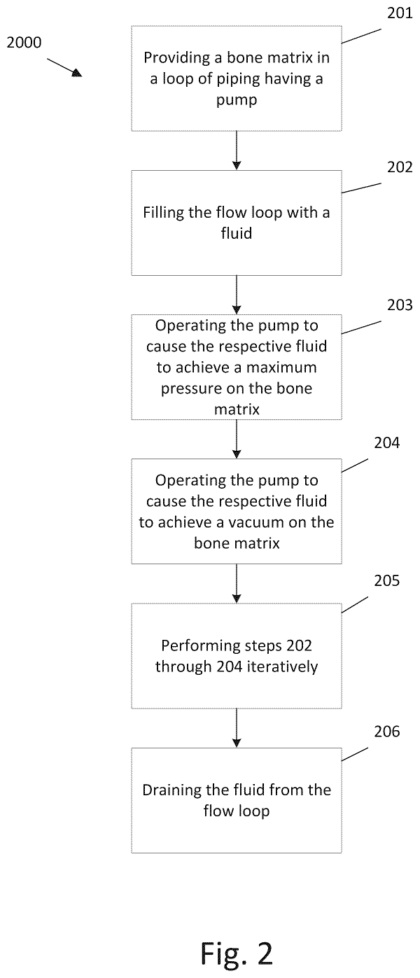

FIG. 2 is a flow chart of an exemplary embodiment of a method 2000 of decellularizing a bone matrix. In an embodiment, at step 201, the method 2000 can include providing a bone matrix in a loop of piping having a pump. At step 202, the method 2000 can include filling the loop of piping with a fluid. At steps 203 through 205, the method 2000 can include iteratively operating the pump in a forward direction to cause the fluid to achieve a positive pressure on the bone matrix (i.e., at step 203) and operating the pump in the backwards direction to cause the fluid to create a vacuum on the bone matrix (i.e., at step 204). In some embodiments, maximum positive pressure can be at least 90 psi. In some embodiments, vacuum is no more than 6 psi. Upon completion of the decellularizing in steps 201-205 above, at step 206, the method 2000 can include draining the fluid from the loop of piping.

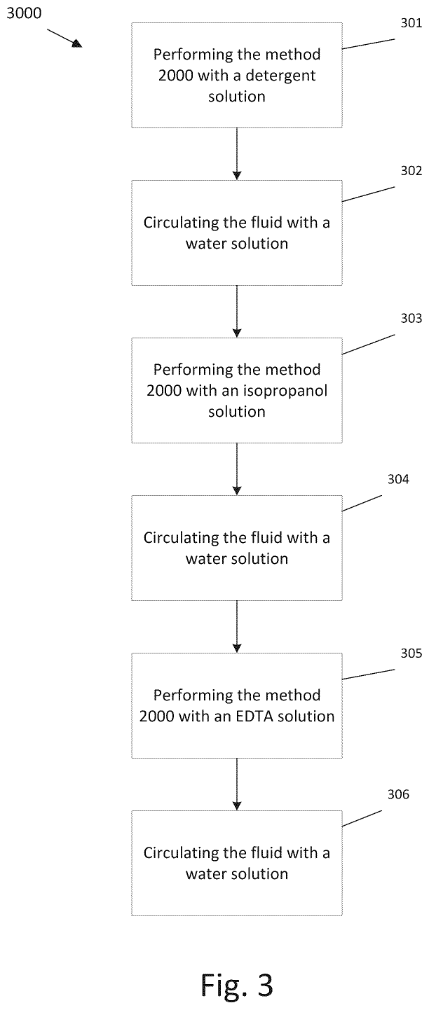

FIG. 3 is a flow chart of an exemplary embodiment of a method 3000 of decellularizing a bone matrix. In an embodiment, the method 3000 can include performing the method 2000 several times, each time with a different solution. For example, in FIG. 3, at step 301, the method 3000 can include performing the method 2000 with a detergent solution. Any type of detergent can be used. In one embodiment, the detergent solution may include a solution of triton-x, tributyl phosphate, and hydrogen peroxide. At step 302, the method 3000 can include circulating a water solution throughout the loop of piping. At step 303, the method 3000 can include performing the method 2000 with an isopropanol or other alcohol solution. At step 304, the method 3000 can include circulating a water solution throughout the loop of piping. At step 305, the method can include performing the method 200 then with an EDTA solution. At step 306, the method can include circulating a water solution throughout the loop of piping. Performing the method 2000 with the water circulation steps 302, 304, and 306 can dilute and remove residual detergent solution, isopropanol solution, or EDTA solution.

In some embodiments, one or more of these steps can be repeated. For example, in some embodiments, steps 301, 302, 304, and 306 can be repeated two or more times. If a step is repeated two or more times, each iteration of the step can be performed with the same solution as the preceding step or with a “fresh” or new solution. With respect to step 301 with the detergent solution, using a fresh detergent solution can reduce the saturation of the detergent solution. Further, each iteration of the step can be performed for the same amount of time as the preceding iteration or a different amount of time.

In an embodiment, the rinse cycle and the wash cycle can each include circulating a water solution through the loop of piping while the AVAP system 1000 is in the recirculation configuration, with the difference between the rinse cycle and the wash cycle being the duration of the cycle. However, the subject matter disclosed herein is not so limited. Instead, the rinse cycle can include circulating a water solution through the loop of piping when the AVAP system 1000 is in the recirculation configuration, and the wash cycle can include performing the method 3000 using a wash solution, which requires the AVAP system 1000 to iteratively be in the vacuum configuration and the pressure configuration. In such embodiments, the method 3000 can further include performing the wash cycle by performing the method 2000 with a water solution before steps 302, 304, and 306.

The functions and process steps herein can be performed automatically or wholly or partially in response to user command. An activity (including a step) performed automatically is performed in response to one or more executable instructions or device operation without user direct initiation of the activity. While various illustrative embodiments incorporating the principles of the present teachings have been disclosed, the present teachings are not limited to the disclosed embodiments. Instead, this application is intended to cover any variations, uses, or adaptations of the present teachings and use its general principles. Further, this application is intended to cover such departures from the present disclosure that are within known or customary practice in the art to which these teachings pertain.

In the above detailed description, reference is made to the accompanying drawings, which form a part hereof. In the drawings, similar symbols typically identify similar components, unless context dictates otherwise. The illustrative embodiments described in the present disclosure are not meant to be limiting. Other embodiments can be used, and other changes may be made, without departing from the spirit or scope of the subject matter presented herein. It will be readily understood that various features of the present disclosure, as generally described herein, and illustrated in the Figures, can be arranged, substituted, combined, separated, and designed in a wide variety of different configurations, all of which are explicitly contemplated herein.

Aspects of the present technical solutions are described herein with reference to flowchart illustrations and/or block diagrams of methods, apparatuses (systems), and computer program products according to embodiments of the technical solutions. It will be understood that each block of the flowchart illustrations and/or block diagrams, and combinations of blocks in the flowchart illustrations and/or block diagrams, can be implemented by computer readable program instructions.

The flowchart and block diagrams in the figures illustrate the architecture, functionality, and operation of possible implementations of systems, methods, and computer program products according to various embodiments of the present technical solutions. In this regard, each block in the flowchart or block diagrams can represent a module, segment, or portion of instructions, which includes one or more executable instructions for implementing the specified logical function(s). In some alternative implementations, the functions noted in the blocks can occur out of the order noted in the figures. For example, two blocks shown in succession can, in fact, be executed substantially concurrently, or the blocks can sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

A second action can be said to be “in response to” a first action independent of whether the second action results directly or indirectly from the first action. The second action can occur at a substantially later time than the first action and still be in response to the first action. Similarly, the second action can be said to be in response to the first action even if intervening actions take place between the first action and the second action, and even if one or more of the intervening actions directly cause the second action to be performed. For example, a second action can be in response to a first action if the first action sets a flag and a third action later initiates the second action whenever the flag is set.

The present disclosure is not to be limited in terms of the particular embodiments described in this application, which are intended as illustrations of various features. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. It is to be understood that this disclosure is not limited to particular methods, reagents, compounds, compositions or biological systems, which can, of course, vary. It is also to be understood that the terminology used herein is for the purpose of describing particular embodiments only, and is not intended to be limiting.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein are generally intended as “open” terms (for example, the term “including” should be interpreted as “including but not limited to,” the term “having” should be interpreted as “having at least,” the term “includes” should be interpreted as “includes but is not limited to,” et cetera). While various compositions, methods, and devices are described in terms of “comprising” various components or steps (interpreted as meaning “including, but not limited to”), the compositions, methods, and devices can also “consist essentially of” or “consist of” the various components and steps, and such terminology should be interpreted as defining essentially closed-member groups.

As used in this document, the singular forms “a,” “an,” and “the” include plural references unless the context clearly dictates otherwise. Unless defined otherwise, all technical and scientific terms used herein have the same meanings as commonly understood by one of ordinary skill in the art. Nothing in this disclosure is to be construed as an admission that the embodiments described in this disclosure are not entitled to antedate such disclosure by virtue of prior invention. As used herein, the terms “about” or “approximately” for any numerical values or ranges indicate a suitable dimensional tolerance that allows the part or collection of components to function for its intended purpose as described herein. More specifically, “about” or “approximately” may refer to the range of values ±20% of the recited value, e.g. “about 90%” may refer to the range of values from 71% to 99%.

In addition, even if a specific number is explicitly recited, those skilled in the art will recognize that such recitation should be interpreted to mean at least the recited number (for example, the bare recitation of “two recitations,” without other modifiers, means at least two recitations, or two or more recitations). Furthermore, in those instances where a convention analogous to “at least one of A, B, and C, et cetera” is used, in general such a construction is intended in the sense one having skill in the art would understand the convention (for example, “a system having at least one of A, B, and C” would include but not be limited to systems that have A alone, B alone, C alone, A and B together, A and C together, B and C together, and/or A, B, and C together, et cetera). In those instances where a convention analogous to “at least one of A, B, or C, et cetera” is used, in general such a construction is intended in the sense one having skill in the art would understand the convention (for example, “a system having at least one of A, B, or C” would include but not be limited to systems that have A alone, B alone, C alone, A and B together, A and C together, B and C together, and/or A, B, and C together, et cetera). It will be further understood by those within the art that virtually any disjunctive word and/or phrase presenting two or more alternative terms, whether in the description, sample embodiments, or drawings, should be understood to contemplate the possibilities of including one of the terms, either of the terms, or both terms. For example, the phrase “A or B” will be understood to include the possibilities of “A” or “B” or “A and B.”

In addition, where features of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, et cetera. As a non-limiting example, each range discussed herein can be readily broken down into a lower third, middle third and upper third, et cetera. As will also be understood by one skilled in the art all language such as “up to,” “at least,” and the like include the number recited and refer to ranges that can be subsequently broken down into subranges as discussed above. Finally, as will be understood by one skilled in the art, a range includes each individual member. Thus, for example, a group having 1-3 components refers to groups having 1, 2, or 3 components. Similarly, a group having 1-5 components refers to groups having 1, 2, 3, 4, or 5 components, and so forth.

Various of the above-disclosed and other features and functions, or alternatives thereof, may be combined into many other different systems or applications. Various presently unforeseen or unanticipated alternatives, modifications, variations or improvements therein may be subsequently made by those skilled in the art, each of which is also intended to be encompassed by the disclosed embodiments.

1. An alternating vacuum and pressure system for decellularizing a bone matrix comprising:
piping having a first end open to atmospheric pressure and a second end open to atmospheric pressure, wherein a subset of the piping forms a loop;
a chamber in fluid communication with the piping, wherein the chamber is configured to contain the bone matrix;
at least one heating element attached to the piping and configured to heat the piping;
a pump; and
a plurality of valves connected to and in fluid communication with the piping,
wherein the plurality of valves can be selectively opened or closed to form one of a plurality of configurations, wherein the plurality of configurations comprises:
a pressure configuration, wherein in the pressure configuration, operation of the pump in a first direction causes a fluid within the piping to travel toward the chamber such that pressure is applied to the chamber, and
a vacuum configuration, wherein in the vacuum configuration, operation of the pump in a second direction causes the fluid within the pipe to travel away from the chamber such that a vacuum is created on the chamber.

2. The system of claim 1, wherein the plurality of valves comprises a loop valve,
wherein closure of the loop valve prevents the fluid within the piping from circulating through the loop, and
wherein the loop valve is closed in the pressure configuration and the vacuum configuration.

3. The system of claim 2, wherein the plurality of valves comprises a pump valve,
wherein closure of the pump valve prevents pumping of a fluid within the piping, and
wherein the pump valve is closed after pressure is established in the pressure configuration and after vacuum is established in the vacuum configuration.

4. The system of claim 3, wherein the plurality of valves further comprises an air valve,
wherein closure of the air valve closes the first end to atmospheric pressure, and
wherein the air valve is closed in the pressure configuration and the vacuum configuration.

5. The system of claim 4, wherein the plurality of valves further comprises a fill valve,
wherein closure of the fill valve prevents a filling fluid from being drawn into the piping, and
wherein the fill valve is closed in the pressure configuration and the vacuum configuration.

6. The system of claim 5, wherein the plurality of valves further comprises a drain valve,
wherein closure of the drain valve closes the second end to atmospheric pressure prevents the fluid within the piping from exiting the piping through the second end, and
wherein the drain valve is closed in the pressure configuration and the vacuum configuration.

7. The system of claim 6, wherein the plurality of configurations further comprises a recirculation configuration,
wherein, in the recirculation configuration, operation of the pump causes fluid to circulate through the loop, and
wherein, in the recirculation configuration, the loop valve and the pump valve are open, and the air valve, the fill valve, and the drain valve are closed.

8. The system of claim 6, wherein the plurality of configurations further comprises a balance configuration, wherein in the balance configuration a pressure of the fluid within the piping will balance,
wherein, in the balance configuration, the air valve, the loop valve, and the pump valve are open, and the fill valve and the drain valve are closed.

9. The system of claim 6, wherein the plurality of configurations further comprises a drain configuration,
wherein, in the drain configuration, the fluid within the piping exits the piping through the second end, and
wherein, in the drain configuration, the air valve, the loop valve, the pump valve, and the drain valve are open.

10. The system of claim 6, wherein the plurality of configurations further comprises a fill configuration, wherein, in the fill configuration, operation of a fill pump causes a filling fluid to travel from a fluid filling source into the piping,
wherein, in the fill configuration, the fill valve, the pump valve, the loop valve, and the air valve are open, and the drain valve is closed.

11. The system of claim 10, wherein a fluid filling source is one of a plurality of tanks comprising:
a water fluid tank comprising water;
an alcohol fluid tank comprising isopropanol;
an acid fluid tank comprising ethylenediaminetetraacetic Acid (EDTA); and
a detergent fluid tank comprising a detergent solution.

12. The system of claim 1 further comprising:
a water fluid tank comprising water;
an isopropanol fluid tank comprising isopropanol;
an ethylenediaminetetraacetic acid (EDTA) fluid tank comprising EDTA; and
a detergent fluid tank comprising a detergent solution.

13. The system of claim 1, wherein the chamber comprises a basket comprising stainless steel mesh, and wherein the basket is configured to contain the bone matrix.

14. The system of claim 13, wherein the chamber further comprises a lid for the basket, wherein the lid comprises stainless steel mesh.

15. The system of claim 1, wherein the at least one heating element comprises a chamber heater configured to heat the chamber.

16. The system of claim 1, wherein the at least one heating element comprises a heating column attached to an exterior surface area of a portion of the piping, wherein the heating column is configured to heat the piping, which will in turn heat the fluid within the piping.

17. A method of decellularizing a bone matrix, the method comprising:
providing a bone matrix in a piping forming a loop, wherein the piping comprises a pump;
performing, for each of a plurality of fluids, a plurality of iterations, wherein each of the plurality of iterations comprises:
filling the loop with a respective fluid of the plurality of fluids,
operating the pump in a first direction to cause the respective fluid to achieve a positive pressure on the bone matrix, and
operating the pump in a second direction to cause the respective fluid to achieve a vacuum on the bone matrix; and
draining the respective fluid from the loop.

18. The method of claim 17, wherein performing, for each of a plurality of fluids, a plurality of iterations comprises:
a first plurality of iterations configured to remove bulk debris from the bone matrix;
a second plurality of iterations to remove lipids from the bone matrix; and
a third plurality of iterations to remove cellular artifacts from the bone matrix.

19. The method of claim 18, wherein the plurality of fluids comprises a detergent solution, a water solution, an alcohol solution, and an ethylenediaminetetraacetic acid (EDTA) solution.

20. The method of claim 19, wherein the performing, for each of the plurality of fluids, the plurality of iterations comprises:
performing, for the detergent solution, the first plurality of iterations;
filling the loop with a first water solution in response to performing the first plurality of iterations;
circulating the first water solution through the loop in response to filling the loop with a first water solution;
draining the first water solution from the loop in response to circulating the first water solution through the loop;
performing, for an isopropanol solution, the second plurality of iterations in response to draining the first water solution;
filling the loop with a second water solution in response to performing the second plurality of iterations;
circulating the second water solution through the loop in response to filling the loop with a second water solution;
draining the second water solution from the loop in response to circulating the second water solution through the loop;
performing, for the EDTA solution, the third plurality of iterations in response to draining the second water solution;
filling the loop with a third water solution in response to performing the third plurality of iterations;
circulating the third water solution through the loop in response to filling the loop with a third water solution; and
draining the third water solution from the loop in response to circulating the third water solution through the loop.
